(19) United States
(12) Patent Application Publication
(10) Pub. No.: US 2011/0061121 A1
(43) Pub. Date: Mar. 10, 2011

(54) SEEDLESS TRIPLOID EGGPLANT

(76) Inventors: Rob Dirks, Oudenbosch (NL); Wilhelmus Petrus Adrianus Roeland Voermans, Etten-Leur (NL); Simone Adriana Maria Oosterwijk, Brielle (NL); Johannes Henrikus Jacobus Van Den Enden, 's-Gravenzande (NL); Jacob Pieter Willem Haanstra, Dordrecht (NL)

(21) Appl. No.: 12/841,344

(22) Filed: Jul. 22, 2010

Related U.S. Application Data
(63) Continuation-in-part of application No. PCT/EP2009/000636, filed on Jan. 30, 2009.

(30) Foreign Application Priority Data
Feb. 1, 2008 (EP) 08001894.8
Nov. 5, 2008 (EP) 08019332.9

(51) Int. Cl. A01H 1/00; A01H 5/00

(52) U.S. Cl. 800/260; 800/317

(57) ABSTRACT

The invention relates to a method for producing a seedless eggplant. The method involves crossing a tetraploid eggplant plant and a diploid eggplant plant to obtain a triploid eggplant seed, and growing a fruit-bearing eggplant plant from the triploid eggplant seed, whereby that fruit is seedless.

### INCORPORATION BY REFERENCE

This application is a Continuation-In-Part of International Application No. PCT/EP2009/000636, filed Jan. 30, 2009, which published as PCT Publication No. WO/2009/095266 on Aug. 6, 2009, which claims benefit of European Patent Application No. 08001894.8, filed Feb. 1, 2008 and European Patent Application No. 08019332.9, filed Nov. 5, 2008.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

The present invention relates to a seedless eggplant and to a method for producing the seedless eggplant.

### BACKGROUND OF THE INVENTION

The eggplant or aubergine (Solanum melongena) is a solanaceous plant bearing a fruit of the same name, commonly used as a vegetable in cooking. It is closely related to the tomato and potato. Because of its large, pendulous, purple or white fruit, the eggplant is an important food crop. It has been cultivated in southern and eastern Asian countries since prehistory but is now also grown in the Western world. The fruit contains numerous small, soft seeds. The presence of seeds in ready-to-harvest fruits is considered to be a negative quality. This is inter alia because seeds may lead to undesired browning of the flesh.

From a commercial point of view, seedlessness is a very desirable trait in edible fruits and vegetables, like the eggplant. In some species, seedlessness is the result of parthenocarpy, where fruit is produced without fertilization. In certain species, parthenocarpic fruit production requires pollination or another stimulus, in particular spraying the flowers with plant growth regulators, such as gibberellin, auxin and cytokinin. This is termed artificial parthenocarpy.

Parthenocarpic eggplant varieties are known (e.g. Talina, Galine). However, during winter cultivation of such eggplant varieties, fruit production may be hampered by suboptimal environmental conditions. These are usually counteracted by treating flower buds with plant growth regulators. However, phytohormonal treatments make the production process more expensive due to the cost of both chemicals and labor. In addition, the use of hormones is under discussion or prohibited in some countries.

Parthenocarpy has also been genetically engineered in eggplant by using the DefH9-iaaM gene. The iaaM gene codes for tryptophan monoxygenase and confers auxin synthesis, while the DefH9 controlling regions drive expression of the gene specifically in the ovules and placenta. This lead to a significant increase in fruit production concomitant with a reduction in cultivation costs. However, in many countries genetically modified crops are not well accepted.

Citation or identification of any document in this application is not an admission that such document is available as prior art to the present invention.

### SUMMARY OF THE INVENTION

It is an object of the present invention to provide a seedless eggplant fruit that does not have the above-stated drawbacks.

The present invention relates to a method for producing a seedless eggplant which may comprise:

crossing a tetraploid eggplant plant and a diploid eggplant plant to obtain a triploid eggplant seed, and

growing a fruit-bearing eggplant plant from the triploid eggplant seed, whereby that fruit is seedless.

In one embodiment, the tetraploid eggplant plant may be produced by doubling the chromosomes of a diploid eggplant seedling or explant. The chromosomes may be doubled by treatment with colchicine.

In another embodiment, the tetraploid eggplant plant may be the mother plant and the diploid eggplant plant may be the father plant.

In yet another embodiment, the tetraploid eggplant plant may be a plant grown from AB5123-tetra seeds, representative seeds of which having been deposited under NCIMB Accession No. 41516.

The present invention is also directed to a seedless eggplant, which may be produced by any one of the methods described above.

The present invention is further directed to a method for producing a seedless eggplant, comprising:

a) growing a tetraploid eggplant plant from AB5123-tetra seeds, representative seeds of which having been deposited under NCIMB Accession No. 41516;

b) crossing the tetraploid eggplant plant with a diploid eggplant plant to obtain a triploid eggplant seed, and

growing a fruit-bearing eggplant plant from the triploid eggplant seed, whereby that fruit is seedless.

In another embodiment, the seedless eggplant plant may be a hybrid.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawing, in which:

FIG. 1 depicts a cutting prepared by taking the hypocotyl from a seedling. The top and all except one leaf of the cutting is removed to prepare explants after treatment with colchicine.

### DETAILED DESCRIPTION

The present invention is directed to a method for producing a seedless eggplant, comprising:

crossing a tetraploid eggplant plant and a diploid eggplant plant to obtain a triploid eggplant seed, and

growing a fruit-bearing eggplant plant from the triploid eggplant seed, whereby that fruit is seedless.

Tetraploid eggplant plants may be produced by doubling the chromosomes of a diploid seedling or explant. In one embodiment, the chromosomes may be doubled by treatment with colchicine.

Chromosome doubling by treatment with colchicine may comprise growing seedlings from eggplant seeds and preparing diploid cuttings therefrom. The cuttings may then be rooted, for example, on an artificial medium. After roots have developed, the top and almost all, preferably all except one, of the other leaves may be removed from the cuttings. The remaining leaf or leaves of the explant may then be contacted with a solution of colchicine for a certain amount of time to effect doubling of the chromosomes. After treatment with the colchicine solution, the cuttings may be grown until shoots develop. These shoots may then be tested for tetraploidy. Diploid shoots may be removed.

In one embodiment, the remaining leaf or leaves may be contacted with a solution of colchicine for about 30 minutes to about 4 hours. In another embodiment, the remaining leaf or leaves may be contacted with a solution of colchicine for about 2 hours.

In one embodiment, ploidy may be determined by flow-cytometry. Tetraploid eggplant plants that are produced may be used as a parent eggplant plant in the production of triploid eggplant plants. In one aspect, the tetraploid eggplant plant may be the mother eggplant plant and the diploid eggplant plant may be the father eggplant plant.

In another aspect, the tetraploid eggplant plant may be a plant grown from AB5123-tetra seeds deposited on 13 Nov. 2007 with the NCIMB in Aberdeen under deposit accession number NCIMB 41516.

The invention may further relate to seedless eggplants produced by the method described above. In one aspect, the seedless eggplant may be produced by:

a) growing a tetraploid eggplant plant from AB5123-tetra seeds, representative seeds of which having been deposited under NCIMB Accession No. 41516;

b) crossing the tetraploid eggplant plant with a diploid eggplant plant to obtain a triploid eggplant seed, and

growing a fruit-bearing eggplant plant from the triploid eggplant seed, whereby that fruit is seedless.

The fruit-bearing eggplant plant of the present invention may produce fruit without pollination or treatment with plant growth regulators.

“Seedless” as used in the present application may refer to the complete or almost complete absence of viable seeds. The fruits may still contain remnants of seeds, such as pellicles or skins.

A “hybrid” of the present invention may refer to a heterozygous offspring. A hybrid plant may carry two different alleles of the same gene, may result from the fusion of gametes that have differing structure in at least one chromosome, or may result from the fusion of gametes having different haploid numbers of chromosomes.

“Ploidy” refers to the number of sets of chromosomes in a biological cell. For example, a plant may be diploid (having two sets of chromosomes), triploid (having three sets of chromosomes), or tetraploid (having four sets of chromosomes).

The invention will now be further described by way of the following non-limiting examples.

### EXAMPLES

### Example 1

### Production of a Tetraploid Eggplant Plant

### Chromosome Doubling by Means of Colchicine

Seeds of a diploid eggplant fruit were sterilized in 0.5% chlorine during 30 min and subsequently rinsed with demineralized water until all foam was removed. Seeds (10 seeds on each plate) were sown on ½MS10 medium. The seeds were germinated at 25° C. under 16 hrs of light. After 2 weeks cuttings were prepared by taking the hypocotyls from the seedlings. These were planted in MS20 medium.

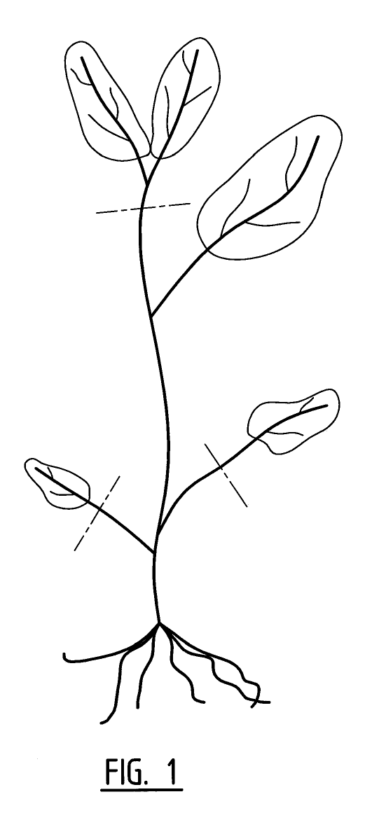

After three weeks, the cuttings were treated with colchicine. The top and all except one leaf of the cuttings were removed according to FIG. 1 to obtain explants. A colchicine solution was prepared by dissolving 5 g colchicine (Sigma C-9754) in 1000 ml water and filter sterilizing. The explants were placed upside down in the solution for 2 hours. The roots did not touch the colchicine solution.

After 2 hours, the explants were rinsed with sterile demineralized water and put on MS20 medium. After they started growing, the plants were potted in soil. Shoots were tested for their ploidy (e.g., tetraploidy) by means of the method as described in De Laat et al., Plant Breeding 99(4), 303-307 (1987).

Diploid shoots were removed.

### Results

The results of various experiments with different parent plants are shown in Table 1.

TABLE 1

| parental | number of | number of | % of tetrapoid |
|---|---|---|---|
| line | potted plants | tetraploid plants | plants |
| T18 | 67 | 40 | 60 |
| T44 | 68 | 37 | 54 |
| T20 | 30 | 25 | 83 |
| T121 | 26 | 13 | 50 |

### Example 2

### Production of Triploid Eggplant Seeds

The seeds obtained from Example 1 were sown under the conditions used by commercial plant raisers. Ploidy levels of the plants obtained was checked by DNA analysis.

Tetraploid plants obtained in this way were used as a mother line for seed production and crossed with a diploid father line in order to reconstruct the original diploid hybrid as a triploid. Seeds obtained from this cross were harvested and plants obtained were checked for ploidy level by flow-cytometry. The results are shown in Table 2. All seeds were found to be triploid.

TABLE 2

| Plant number | Ploidy | Remarks |
|---|---|---|
| 401-1 | 2× | 401 = diploid parent |
| 401-2 | 2× | |
| 401-3 | 2× | |
| 401-4 | 2× | |
| 402-1 | 4× | 402 = tetraploid version of |
| 402-2 | 4× | 401 |
| 402-3 | 4× | |
| 402-4 | 4× | |
| 402 offtype | 2× | |
| 403-1 | 2× | 403 = diploid parent |
| 403-2 | 2× | |
| 403-3 | 2× | |
| 403-4 | 2× | |
| 404-1 | 4× | 404 = tetraploid parent of |
| 404-2 | 4× | 403 |
| 404-3 | 4× | |
| 404-4 | 4× | |
| Fantastic-1 | 2× | hybrids from a cross with a |
| Fantastic-2 | 2× | 401 mother |
| Fantastic-3 | 2× | |
| Fantastic-4 | 2× | |
| Fantastic3n-1 | 3× | hybrids from a cross with a |
| Fantastic3n-2 | 3× | 402 mother |
| Fantastic3n-3 | 3× | |
| Fantastic3n-4 | 3× | |
| Monarca-1 | 2× | hybrids from a cross with a |
| Monarca-2 | 2× | 403 mother |
| Monarca-3 | 2× | |
| Monarca-4 | 2× | |
| Monarca3n-1 | 3× | hybrids from a cross with a |
| Monarca3n-2 | 3× | 404 mother |
| Monarca3n-3 | 3× | |
| Monarca3n-4 | 3× | |

### Example 3

### Analysis of Triploid Eggplant Hybrid and its Parents

Triploid hybrid seeds obtained from Example 2 were sown under the conditions used by commercial plant raisers and planted in a hybrid testing field in autumn in Almeria, Spain. The ploidy level of the hybrid plants obtained was checked by DNA analysis. All hybrid plants were found to be triploid. Fruit set and fruit quality of the triploid hybrid plants Fantastic3n and Monarca3n were analyzed and compared with the original diploid hybrid plants Fantastic and Monarca (Table 2). The results are shown in Table 3. Table 4 shows the same comparison between the diploid parents 401 and 403 and the tetraploid parents 402 and 404 derived therefrom.

From the tables it follows that the fruits of the triploid hybrids are comparable to the fruits of the original diploid mother. The same applies to the tetraploid mother plants.

TABLE 3

| harvested | Fantastic 2n | | Fantastic 3n | | Monarca 2n | | Monarca 3n | |
|---|---|---|---|---|---|---|---|---|
| 2 Jan. 2008 | | | | | | | | |
| number of fruits | 2 | fr | 0 | | 1 | fr | 4 | fr |
| fruit length | 19 | cm | 0 | | 23 | cm | 20 | cm |
| fruit diam | 11 | cm | 0 | | 12 | cm | 12 | cm |
| color | black | | 0 | | black | | black | |
| fruit weight | 360 | g | 0 | | 536 | g | 384 | g |
| plant length | 85 | cm | 95 | cm | 84 | cm | 78 | cm |
| internode length | 8 | cm | 7.5 | cm | 13 | cm | 8 | cm |
| leaf width | 22 | cm | 21 | cm | 18 | cm | 23 | cm |
| leaf length | 32 | cm | 30 | cm | 33 | cm | 28 | cm |
| 8 Jan. 2008 | | | | | | | | |
| number of fruits | 7 | fr | 4 | fr | 2 | fr | 0 | |
| fruit length | 21 | cm | 18 | cm | 22 | cm | 0 | |
| fruit diameter | 10 | cm | 12 | cm | 10 | cm | 0 | |
| color | black | | black | | black | | | |
| fruit weight | 330 | g | 370 | g | 390 | g | 0 | |
| plant length | 92 | cm | 97 | cm | 93 | cm | 83 | cm |
| internode length | 10 | cm | 9 | cm | 13 | cm | 8 | cm |
| leaf width | 21 | cm | 21 | cm | 18 | cm | 23 | cm |
| leaf length | 31 | cm | 33 | cm | 33 | cm | 28 | cm |
| 15 Jan. 2008 | | | | | | | | |
| number of fruits | 0 | | 2 | fr | 2 | fr | 2 | fr |
| fruit length | 0 | | 19.5 | cm | 23 | cm | 23 | cm |
| fruit diameter | 0 | | 10 | cm | 8 | cm | 7 | cm |
| color | 0 | | black | | black | | black | |
| fruit weight | 0 | | 425 | g | 330 | g | 445 | g |
| plant length | 95 | cm | 97 | cm | 94.5 | cm | 87 | cm |
| internode length | 10 | cm | 9 | cm | 13 | cm | 8 | cm |
| leaf width | 22 | cm | 21 | cm | 18 | cm | 23 | cm |
| leaf length | 31 | cm | 33 | cm | 33 | cm | 28 | cm |
| 24 Jan. 2008 | | | | | | | | |
| number of fruits | 4 | fr | 2 | fr | 2 | fr | 3 | fr |
| fruit length | 23 | cm | 20 | cm | 22 | cm | 23 | cm |
| fruit diameter | 8 | cm | 11 | cm | 9 | cm | 8 | cm |
| color | black | | black shiny | | black shiny | | black shiny | |
| fruit weight | 355 | g | 322 | g | 383 | g | 417 | g |
| plant length | 97.5 | cm | 105 | cm | 96 | cm | 87.5 | cm |
| internode length | 10.5 | cm | 9 | cm | 13 | cm | 9 | cm |
| leaf width | 23 | cm | 22 | cm | 18 | cm | 24 | cm |
| leaf length | 32 | cm | 33 | cm | 33.5 | cm | 30 | cm |

TABLE 4

| Harvested | 401 (2n) | | 402 (4n) | | 403 (2n) | | 404 (4n) | |
|---|---|---|---|---|---|---|---|---|
| 2 Dec. 2007 | | | | | | | | |
| number of fruits | 0 | | 2 | fr. | 0 | | 0 | |
| fruit length | 0 | | 14 | cm | 0 | | 0 | |
| fruit diameter | 0 | | 10 | cm | 0 | | 0 | |
| color | — | | black shiny | | 0 | | 0 | |
| fruit weight | — | | 346.5 | g | 0 | | 0 | |
| plant length | 137 | cm | 127.5 | cm | 145 | cm | 80 | cm |
| internode length | 15 | cm | 12 | cm | 17 | cm | 11 | cm |
| leaf width | 18 | cm | 25 | cm | 18 | cm | 20 | cm |
| leaf length | 34 | cm | 32 | cm | 33 | cm | 28 | cm |
| 12 Dec. 2007 | | | | | | | | |
| number of fruits | 4 | fr | 3 | fr | 2 | fr | 0 | |
| fruit length | 17 | cm | 16 | cm | 22 | cm | 0 | |
| fruit diameter | 11 | cm | 14 | cm | 7.5 | cm | 0 | |
| color | black shiny | | sensitive | | black | | 0 | |
| | | | skin/black | | | | | |
| fruit weight | 290 | g | 415 | g | 355 | g | 0 | |
| plant length | 139 | cm | 128 | cm | 153 | cm | 86 | cm |
| internode length | 16 | cm | 11 | cm | 16.5 | cm | 11 | cm |
| leaf width | 20 | cm | 24 | cm | 26 | cm | 20 | cm |
| leaf length | 38 | cm | 33 | cm | 38 | cm | 26 | cm |
| 19 Dec. 2007 | | | | | | | | |
| number of fruits | 6 | fr | 1 | fr | 1 | fr | 0 | |
| fruit length | 19 | cm | 19 | cm | 22 | cm | 0 | |
| fruit diameter | 9 | cm | 12 | cm | 8 | cm | 0 | |
| color | black | | black | | black | | 0 | |
| fruit weight | 375 | g | 470 | g | 370 | g | 0 | |
| plant length | 140 | cm | 130 | cm | 153 | cm | 86 | cm |
| internode length | 15 | cm | 13 | cm | 17 | cm | 11 | cm |
| leaf width | 18 | cm | 25 | cm | 18 | cm | 20 | cm |
| leaf length | 34 | cm | 29 | cm | 34 | cm | 26 | cm |

| 2 Jan. 2008 | | | | | | | | |
|---|---|---|---|---|---|---|---|---|
| number of fruits | 3 | fr | 2 | fr | 0 | | 1 | fr |
| fruit length | 22 | cm | 18 | cm | 0 | | 22 | cm |
| fruit diameter | 12 | cm | 12 | cm | 0 | | 9 | cm |
| color | slightly | | dark black | | 0 | | black | |
| | purple | | | | | | | |
| fruit weight | 440 | g | 440 | g | 0 | | 458 | g |
| plant length | 143 | cm | 130 | cm | 155 | cm | 101 | cm |
| internode length | 15 | cm | 12 | cm | 17 | cm | 11 | cm |
| leaf width | 20 | cm | 25 | cm | 18 | cm | 20 | cm |
| leaf length | 34 | cm | 28 | cm | 34 | cm | 26 | cm |

The invention is further described by the following numbered paragraphs:

1. A method for producing a seedless eggplant, comprising:

crossing a tetraploid eggplant plant and a diploid eggplant plant to obtain a triploid eggplant seed, and

growing a fruit-bearing eggplant plant from the triploid eggplant seed, whereby that fruit is seedless.

2. The method of paragraph 1, wherein the tetraploid eggplant plant is produced by doubling the chromosomes of a diploid eggplant seedling or explant.

3. The method of paragraph 2, wherein the chromosomes of the diploid eggplant seedling or explant are doubled by treatment with colchicine.

4. The method of any one of paragraphs 1-3, wherein the tetraploid eggplant plant is the mother plant and the diploid eggplant plant is the father plant.

5. The method of claim 1, wherein the tetraploid eggplant plant is grown from AB5123-tetra seeds, representative seeds of which having been deposited under NCIMB accession no. 41516.

6. A seedless eggplant obtained by the method of any one of paragraphs 1-5.

7. A method for producing a seedless eggplant, comprising:

a) growing a tetraploid eggplant plant from AB5123-tetra seeds, representative seeds of which having been deposited under NCIMB accession no. 41516;

b) crossing the tetraploid eggplant plant with a diploid eggplant plant to obtain a triploid eggplant seed, and

growing a fruit-bearing eggplant plant from the triploid eggplant seed, whereby that fruit is seedless.

8. The seedless eggplant of paragraph 7.

9. The seedless eggplant of paragraph 6 or 7, wherein the seedless eggplant is a hybrid.

10. A method for producing a seedless eggplant comprising the steps of:
a) providing a first tetraploid eggplant parent plant;
b) providing a second eggplant parent plant that is diploid;
c) crossing the first and the second parent plant to obtain a population of progeny plants; and
d) selecting a triploid plant from the population of progeny plants as the seedless eggplant.

11. The method of paragraph 10, wherein the tetraploid eggplant parent plant is produced by doubling the chromosomes of a diploid eggplant seedling or explant.

12. The method of paragraph 11, wherein the chromosomes are doubled by treatment with colchicine.

13. The method of any one of paragraphs 10-12, wherein the tetraploid parent is the mother plant and the diploid parent is the father plant.

14. The method of paragraph 10, wherein the tetraploid parent plant is a plant grown from AB5123-tetra seeds, representative seeds of which having been deposited under NCIMB accession no. 41516.

15. A seedless eggplant obtainable by performing the method of any one of paragraphs 10-14.

16. A seedless eggplant obtained by:
a) growing a first parent plant from AB5123-tetra seeds, representative seeds of which having been deposited under NCIMB accession no. 41516;
b) crossing the first parent plant with a second diploid eggplant parent plant to obtain a population of progeny plants;
c) selecting the triploid plants from the population of progeny plants as the seedless eggplant.

17. The seedless eggplant of paragraph 6 or 7, which eggplant is a hybrid.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

Each patent, patent application, and publication cited or described in the present application is hereby incorporated by reference in its entirety as if each individual patent, patent application, or publication was specifically and individually indicated to be incorporated by reference.

1. A method for producing a seedless eggplant comprising the steps of:
a) providing a first tetraploid eggplant parent plant;
b) providing a second eggplant parent plant that is diploid;
c) crossing the first and the second parent plant to obtain a population of progeny plants; and
d) selecting a triploid plant from the population of progeny plants as the seedless eggplant.

2. The method of claim 1, wherein the tetraploid eggplant parent plant is produced by doubling the chromosomes of a diploid eggplant seedling or explant.

3. The method of claim 2, wherein the chromosomes are doubled by treatment with colchicine.

4. The method of claim 1, wherein the tetraploid parent is the mother plant and the diploid parent is the father plant.

5. The method of claim 1, wherein the tetraploid parent plant is a plant grown from AB5123-tetra seeds, representative seeds of which having been deposited under NCIMB accession no. 41516.

6. A seedless eggplant obtained by the method of claim 1.

7. A seedless eggplant obtained by:
a) growing a first parent plant from AB5123-tetra seeds, representative seeds of which having been deposited under NCIMB accession no. 41516;
b) crossing the first parent plant with a second diploid eggplant parent plant to obtain a population of progeny plants;
c) selecting a triploid plant from the population of progeny plants as the seedless eggplant.

8. The seedless eggplant of claim 6 or 7, which eggplant is a hybrid.

9. A method for producing a seedless eggplant, comprising:
crossing a tetraploid eggplant plant and a diploid eggplant plant to obtain a triploid eggplant seed, and
growing a fruit-bearing eggplant plant from the triploid eggplant seed,
whereby that fruit is seedless.

10. The method of claim 9, wherein the tetraploid eggplant plant is produced by doubling the chromosomes of a diploid seedling or explant.

11. The method of claim 10, wherein the chromosomes of the diploid seedling or explant are doubled by treatment with colchicine.

12. The method of claim 9, wherein the tetraploid eggplant plant is the mother plant and the diploid eggplant plant is the father plant.

13. The method of claim 10, wherein the tetraploid eggplant plant is the mother plant and the diploid eggplant plant is the father plant.

14. The method of claim 11, wherein the tetraploid eggplant plant is the mother plant and the diploid eggplant plant is the father plant.

15. The method of claim 9, wherein the tetraploid eggplant plant is grown from AB5123-tetra seeds, representative seeds of which having been deposited under NCIMB Accession No. 41516.

16. A seedless eggplant obtained by the method of claim 9.

17. A method for producing a seedless eggplant, comprising:
a) growing a tetraploid eggplant plant from AB5123-tetra seeds, representative seeds of which having been deposited under NCIMB Accession No. 41516;
b) crossing the tetraploid eggplant plant with a diploid eggplant plant to obtain a triploid eggplant seed, and
c) growing a fruit-bearing eggplant plant from the triploid eggplant seed,
whereby that fruit is seedless.

18. A seedless eggplant from the method of claim 17.

19. The seedless eggplant of claim 16 or 18, wherein the seedless eggplant is a hybrid.
